(19) United States
(12) Patent Application Publication
(10) Pub. No.: US 2003/0215479 A1
(43) Pub. Date: Nov. 20, 2003

(54) Process for manufacture of hair or skin cosmetic products using apparatuses with microstructured units

(76) Inventors: Gerhard Sendelbach, Darmstadt (DE); Walter Keller, Ober-Ramstadt (DE); Hans Joachim Spika, Bickenbach (DE); Gerlinde Gockeln, Burghaun (DE); Christian Springob, Lorsch (DE); Juergen Allwohn, Burgschwalbach (DE)

Correspondence Address: STRIKER, STRIKER & STENBY, 103 East Neck Road, Huntington, NY, 11743, US

(21) Appl. No.: 10/426,095

(22) Filed: Apr. 29, 2003

(30) Foreign Application Priority Data
May 2, 2002 (DE) 102 19 523.4

(51) Int. Cl. A61K 7/00

(52) U.S. Cl. 424/401

(57) ABSTRACT

A process for manufacturing hair cosmetic or skin cosmetic products is described, in which the products contain a preparation with hair cosmetic or skin cosmetic ingredients and one process step includes conducting at least a part of the preparation through an apparatus with microstructured units. The apparatus with microstructured units can be one or more static micromixers and/or one or more microheat exchangers.

### BACKGROUND OF THE INVENTION

1. Field of the Invention

The subject matter of the present invention includes a process for production or manufacture of hair or skin cosmetic products, especially of aqueous emulsions or suspensions, which contain at least one skin cosmetic or hair cosmetic ingredient, using apparatuses with microstructured units.

2. Description of the Related Arts

Dispersed preparations of emulsions or suspensions play an outstanding roll in the manufacture of hair and skin cosmetics. Typical products are, for example, hair care compositions, hair dye creams, sunscreen compositions, skin care creams, pigment-containing products, etc. The quality, consistency, effectiveness of the final products and the efficiency of the production process are thus dependent to a large extent on the type of manufacturing process used, especially on the homogenizing process used. An overview of modern processes for manufacturing of semisolid and liquid emulsions is described in the article in SÖFW Journal, 124^{th} Year, 5/98, pp. 308 to 313 and the article in SÖFW Journal, 118^{th} Year, 5/92, pp. 287 to 296. The various processes can be differentiated in hot/hot-, hot/cold- and cold/cold-methods in regard to temperature control. The standard process for emulsion production is the hot/hot-process, in which the fatty phase is heated to about 75° C. and is combined with a hot water phase similarly at about 75° C. Subsequently removal of excess energy supplied in the form of heat occurs with considerable consumption of time. Homogenizers with rapidly rotating rotors are usually used for homogenization. In these devices the phases to be dispersed are broken up into the smallest particles or fractions. The cosmetic emulsions or suspensions conventionally made in this manner do not always have satisfactory cosmetic properties. They frequently have the disadvantage that the emulsified or suspended particles have too non-uniform and too large a particle size and, connected with that, too little effective surface area for many applications. Also they are not distributed optimally in the emulsion and/or suspension. Inhomogeneities can occur because of selective precipitation of dispersed wax material due to poorly controlled cooling. Moreover high temperatures can be disadvantageous for temperature-sensitive or volatile ingredients. Furthermore there is a danger that oxygen will be stirred in during homogenizing, which can damage oxidation sensitive materials, for example the dyeing results and product stability of oxidation dye pre-cursor compositions for oxidation hair dyeing. Furthermore relatively high emulsifier amounts are required for achieving the desired viscosities. Comparatively high emulsifier amounts can have a negative influence on the skin compatibility.

### SUMMARY OF THE INVENTION

It is an object of the present invention to improve the quality, consistency and effectiveness of the hair and skin cosmetic products, especially of multi-phase, dispersion type products.

It is also an object of the present invention to provide improved processes for manufacturing the hair and skin cosmetic products that avoid the above-described disadvantages or at least to reduce them.

According to the invention the process for manufacturing hair cosmetic or skin cosmetic products, which include a preparation that contains at least one hair cosmetic or skin cosmetic ingredient, comprises at least one process step, which includes conducting the preparation, or at least one part of the preparation, through an apparatus with microstructured units. The amount of the hair or skin cosmetic ingredient in the preparation is between 0.05 to 50 percent by weight depending on the type of cosmetic ingredient and the intended action.

The term “manufacturing process” means a process according to the invention, in which products are not made immediately prior to use by the end user, but are made in large quantities by an industrial producer or manufacturer. Especially the preparation is made in an amount, which exceeds that required for a single application, and which especially is sufficient for fulfilling more than one, i.e. a plurality of, final packaging units. Typical production rates are at least 5 kg/h, preferably at least 10 kg/h, especially preferably 50 kg/h.

Especially the preferred microstructured units are micriomixers or microheat exchangers. The microstructured units are fine structured bodies, whose manufacture is described for example in EP 0 391 895. They are assembled from several layer members placed over each other, in which canal-like channels or passages of high stability, high accuracy and surface quality are provided. These layer members can be metal foils of a thickness of from 10 to 1000 μm. The average diameter of the channels amounts preferably to 1000 μm at maximum and is especially preferably from 10 to 1000 μm. The height and depths of the channels preferably are less than 1000 μm, especially preferably less than 250 μm. The wall thickness between the channels and at the channel bottom is preferably less than 100 μm, especially preferably less than 70 μm. Generally the micromixer preferably has at least 1000 microchannels and comprises a stack of foils with several channels extending through them. The apparatus is designed in regard to parallel operating micromixers, the number and diameter of the microchannels and the connected pumps so that the throughput of preferably at least 5 kg/h, especially preferably at least 50 kg/h, is guaranteed. The production process according to the invention is however suitable for manufacture of 100 kg/h to 1000 kg/hr and more, i.e. a daily production of 10 tons and more.

Suitable micromixers are described in EP 758 917, EP 857 080, EP 1 187 671 and DE 197 03 779. Suitable microheat exchangers are described in EP 0 391 895 or in EP 1 046 867. The micromixers and microheat exchangers described in these references are components or parts of the present invention. Suitable micromixers have several channels above and/or next to each other with a diameter of from 10 to 1000 μm, which can be arranged above each other in several layers. Different phases to be thoroughly mixed flow through the channels. The mixing occurs at the outlet of the phases from the channels. To increase the capacity of the method according to the invention the number of channels are increased in the layer members arranged above each other or the number of layer members can be increased or several micromixers can be operated in a module like manner connected in parallel. Also two or more micromixers can be operated in series with each other. It is particularly preferred, when a gross mixing is produced first with a micromixer with a comparatively large channel diameter and following micromixers have increasingly small channel diameters.

When a heating of at least one of the components to be mixed and/or a cooling of the final product is required in addition to a thorough mixing, the heating and/or cooling occurs preferably by conducting through at least one microheat exchanger. The microheat exchanger comprises a plurality of channels arranged over and/or next to each other with diameters of preferably from 10 to 1000 μm. A heat-transfer medium, on one side, and a phase to be mixed and/or the mixed preparation, on another side, alternately flow through spatially neighboring channels.

One embodiment of the process according the invention is a process for manufacturing a dispersion-type hair cosmetic or skin cosmetic preparation, in which

a first partial composition and a second partial composition immiscible with the first partial composition are fed separately to a micromixer, i.e. each to a respective portion of the microchannels of the micromixer, or jointly as a pre-mixture to the micromixer;

the phases are mixed during condition through the micromixer;

at least one hair cosmetic or skin cosmetic ingredient is contained in one of the partial compositions or is added to the mixed preparation, after they flow out of the micromixer.

After output from the micromixer the homogenized preparation can be filled directly into the final or end packaging unit. In case mixing occurred with heating, the homogenized composition is cooled prior to filling, which occurs preferably by conducting through a microheat exchanger. The dissipated heat can be fed back into the manufacturing process. In case introduction of further ingredients is required in the homogenized composition, this can subsequently occur by means of a conventional stirring or dispersion tool or by the use of other micromixers.

If at least one of the phases to be homogenized is a liquid, the second and if necessary additional phases can be liquid, solid or gaseous. With the process according to the invention the two phases to be mixed are conducted together into a micromixer so that the ingredients are mixed in the mixing zone at the outlets of the feed channels. The hair cosmetic or skin cosmetic ingredients can be a hair care ingredient, a hair dye ingredient, a hair fixing ingredient, an ingredient with a light protecting action for skin and/or hair, a perfume, a skin care ingredient, an antidandruff ingredient, a hair and/or skin cleansing ingredient or a preservative. Typical effective ingredient amounts are thus 0.05 to 20, preferably from 0.14 to 10, percent by weight.

The portion of the phase to be homogenized in the finished emulsion or suspension conforms to the requirements of the final product to be manufactured. The lipophilic phase for hair care agents can, for example, be from 2 to 10 percent by weight or for creams, such as hair dye creams, even up to 50 percent by weight. The homogenizing can occur emulsion-free. An emulsifier or surfactant can also be present as a dispersing-assisting agent. The dispersing-assisting agent can be present in the finished composition in an amount of from 0.5 to 30 percent by weight. Nonionic, anionic, cationic, amphoteric or zwitterionic emulsifiers are suitable as the emulsifier in the present invention. Suitable emulsifiers are, for example, found in “International Cosmetic Ingredient Dictionary and Handbook”, Chapter 7, Volume 2, in the section “Surfactants”, especially in the subsection “Surfactants—Emulsifying Agents”. Nonionic emulsifiers include e.g. ethoxylated fatty alcohols, ethoxylated nonylphenols, fatty acid mono and diglycerides, ethoxylated and hydrogenated or nonhydrogenated castor oil, fatty acid alkanol amides, ethoxylated fatty acid ester. Cationic emulsifiers include e.g. long chair quaternary ammonium compounds like those known under the CTFA name “Quaternium”, such as alkyltrimethyl ammonium salts or dialkyldimethyl ammonium salts with C₈- to C₂₂-alkyl groups. Anionic emulsifiers include, e.g., fatty alcohol sulfates, alkyl ether sulfates, alkylbenzene sulfonates, and amphoteric emulsifiers include, e.g., betaines, such as fatty acid amide alkylbetaines and sulfobetaines and C₈- to C₂₂-alkylbetaines.

Other substances, which are usually added as turbidity-inducing agents in cosmetic compositions, are suitable as substances to be homogenized. These other substances include those of formula R¹—COO—(CHR⁴CHR⁵O)ₙ-COR⁶, wherein R¹ represents a C₈- to C₂₂-alkyl group, R⁴ and R⁵ represent hydrogen or methyl and R⁶ represents hydrogen or R¹, and n is a whole number between 1 and 12, preferably 1, 2, 3 or 4. Glycol difatty acid esters are especially preferred. The process according to the invention is especially suitable for manufacture of a turbidity-inducing agent preparation for cosmetic compositions. To accomplish this first a concentrated alkyl ether sulfate, e.g. lauryl ether sulfate, is dissolved in unheated, electrolyte-free water by means of a micromixer. Subsequently a water-insoluble turbidity-inducing agent, e.g. an ethylene or polyethylene glycol difatty acid ester, such as polyethylene glycol-(3)-distearate is homogenized in a liquid or melted state by means of a micromixer.

Another embodiment concerns the manufacture of cosmetic O/W- or W/O emulsions, which are made in a so-called hot/hot process in such a way that both the aqueous and the hydrophobic phases are heated and emulsified in the heated state. In one embodiment according to the invention an aqueous liquid phase is mixed with a liquid hydrophobic phase in a micromixer. The micromixer has channels with a diameter of 10 to 1000 μm. At least one of the phases is fed to a microheat exchanger for heating prior to pre-mixing and/or the mixed preparation is fed to a microheat exchanger for cooling. The microheat exchanger comprises several channels, which are adjacent and/or above each other with diameters of preferably 10 to 1000 μm. A heat transfer medium on one side and one of the phases and/or the mixed preparation on the other side flow through alternate spatially adjacent channels of the microheat exchanger. The phrase “aqueous phase” includes water and mixtures of water with soluble solvents, such as lower alcohols, e.g. ethanol or isopropanol, or polyols, such as ethylene glycol, diethylene glycol, butylene glycol or glycerol.

It is particularly advantageous to perform hot/cold or cold/cold processes instead of the conventional hot/hot process. An aqueous liquid phase is emulsified with a hydrophobic liquid phase for this purpose. In these processes either none of the phases (cold/cold emulsifying) or only one phase (hot/cold emulsifying) is heated. Heating is understood to mean a temperature above room temperature (25° C.), preferably greater than 40° C. If the hydrophobic phase is present as a solid at room temperature, as is the case with a wax or waxy substance, this substance is added to the micromixer in the melted state. The aqueous phase is fed to the micromixer with a temperature that is less than or equal to room temperature in a hot/cold process according to the invention.

When the final product is a viscous composition, the desired final viscosity, especially, when it is an O/W emulsion, is already frequently produced by the homogenization. The desired final viscosity however can also be adjusted by addition of an electrolyte, such as NaCl, or another thickening ingredient, such as cellulose or cellulose derivative.

The particle diameter of the dispersed phase is preferably less than 1 μm, especially preferably less than 0.2 μm. In an additional embodiment the channel dimensions of the microstructures of the micromixer and the flow and pressure ratios are selected so that a nanoemulsion of aqueous and hydrophobic phases results. A nanoemulsion is an emulsion with particle sizes amounting to 100 nm or less.

The processes according to the invention can be performed batchwise or continuously. The continuous process is particularly preferred, especially, when heating of at least one component and cooling is required after mixing and cooling by means of a microheat exchanger occurs. The subject matter according to the invention thus includes a continuous process for manufacturing a dispersion-type hair cosmetic or skin cosmetic preparations, in which a preparation that is heated and dispersed in a micromixer is fed to a microheat exchanger after dispersion and cooled there and subsequently the preparation is filled into a portioned container. The continuous process is especially advantageous when the hydrophobic phase contains a meltable fat or wax material that is solid at room temperature. This latter fat or wax material is melted prior to mixing, is dispersed in an aqueous phase in the melted state. Subsequently the resulting dispersion is subsequently cooled to a temperature under the melting point of the fat or wax material by means of a microheat exchanger. Then it is filled into a container immediately. In case the hydrophobic phase is present at room temperature completely in liquid form, the dispersing can also occur completely without heating.

The dispersing of an aqueous phase with an immiscible hydrophobic phase can occur according to the inventive process with or without an emulsifier. A special advantage of the process is that substantially less emulsifier needs to be consumed in order to obtain a stable emulsion or dispersion of a certain viscosity. For that reason the sensitization potential is reduced and the skin compatibility is improved. If the use of an emulsifier is completely eliminated, metastable dispersions form with lengthened stability in contrast to the dispersions made according to conventional processes. The subject matter of the invention thus includes a process for manufacture of a dispersion-type hair cosmetic or skin cosmetic preparation, in which a hydrophobic phase is mixed in a micromixer without emulsifier with an aqueous phase.

Additionally the subject matter of the invention includes a process for manufacturing a hair cosmetic or skin cosmetic preparation, which contains at least one temperature-sensitive substance, in which the preparation, the at least one temperature-sensitive substance or a partial preparation for heating or cooling is conducted through a microheat exchanger. The temperature sensitive substance can be, e.g., an easily volatilized substance, a substance decomposing at temperatures above room temperature or a substance which reacts chemically with at least one of the other ingredients above room temperature. This is particularly advantageous during manufacture of perfumed hair or skin cosmetics using easily volatilized perfume or fragrance ingredients. Completely different effects, such as a very intense perfume effect, can be achieved in the final product using apparatuses with microstructured units.

Suitable easily-volatilized substances are liquid at room temperature and preferably have boiling points in a range of from 30 to 250° C., especially preferably from 60 to 220° C. For example, liquid hydrocarbons, liquid cyclic or linear silicones (dimethylpolysiloxanes) or mixtures thereof are suitable as the easily-volatilized substances. Suitable hydrocarbons are paraffins or isoparaffins with 5 to 14 C-atoms, especially preferably with 8 to 12 carbon atoms. The suitable hydrocarbons include pentane, hexane, dodecane or isododecane. Suitable liquid, easily volatilized silicones include cyclic dimethylsiloxanes with 3 to 8, preferably 4 to 6, silicon atoms, especially cyclotetradimethylsiloxane, cyclopentadimethylsiloxane or cyclohexadimethylsiloxane. Dimethyl-siloxane/methylalkylsiloxane cyclocopolymers, e.g. Silicone FZ 3109 of Union Carbide, which is a dimethylsiloxane/methyloctylsiloxane cyclocopolymer, is especially suitable. Suitable volatile linear silicones have 2 to 9 silicon atoms. For example, hexamethyldisiloxanes or alkyltrisiloxanes, such as hexylheptamethyltrisiloxanes or octylheptamethyltrisiloxanes, are suitable.

The subject matter of the invention also includes a process for manufacturing of hair wax products, in which the final product has a waxy or wax-like consistency at room temperature (25° C.). This final product contains either at least two different waxy effective ingredients or at least one waxy effective ingredient and at least one other, non-waxy additive or effective ingredient. In this process the thorough mixing of the ingredients occurs in a heated liquid state and the mixed heated and liquid composition is conducted through a microheat exchanger for heat dissipation. The terms “wax”, “waxy”, “wax-like” are related especially to the definition of “wax” given in Ullmanns' Encyclopedia of Industrial Chemistry, 4th Edition, Volume 24, p. 3 (Ullmanns Encykopädie der technischen Chemie, 4. Auflage, Band 24, Seite 3). According to this reference “waxes” are kneadable at 20° C., solid to brittle hard, coarse to fine crystalline and translucent to opaque. However they are not glassy, melt without decomposition above 40° C. and have comparatively low viscosity immediately above their melting point. They have consistencies and viscosities that are comparatively strongly temperature dependent and can be polished under gentle pressure. The wax-like or waxy composition of the hair wax product has at 25° C. a needle penetration number of preferably greater than or equal to 10, especially preferably greater than or equal to 20. The following measurement conditions were used to obtain these needle penetration numbers: measurement unit 0.1 mm, test weight 100 g, test duration 5 s, test temperature 25° C.; according to DIN 51579. The preparation according to the invention contains 20 to 60 percent by weight, especially preferably 30 to 50, percent by weight of the waxy or wax-like substance. It can also contain liquid hydrophobic oils, preferably in an amount of 10 to 35 percent by weight, especially preferably 15 to 30 percent by weight. Moreover easily volatilized hydrophobic substances are contained in an amount of preferably 10 to 35, especially preferably from 15 to 30, percent by weight. The term “easily-volatilized materials” means, within the context of the present invention, those materials that evaporate from the hair when they are applied to the hair. The pointing point of these substances is usually about 250° C. or less.

Each of the waxes known in the state of the art can in principle be used as the wax or waxy materials in the hair wax product according to the invention. For this purpose there are numerous natural waxes, especially animal and plant wax, such as beeswax, wool wax and its derivatives, such as wool wax alcohols, candelilla wax, carnauba wax, Japan wax; fossil wax, especially mineral waxes; synthetic waxes; partially synthetic waxes; microcrystalline waxes; macrocrystalline waxes; hydrocarbon waxes, especially solid paraffin or petrolatum; oxygen functionalized hydrocarbon waxes; fatty substances, such as hardened fats, fatty acids, fatty alcohols, fatty acid esters, fatty alcohol esters or fatty acid glycerides, in which the named fatty substances preferably have at least 12 carbon atoms; polyalkylene glycol waxes, especially polyethylene glycol waxes; silicone waxes; and mono- or diesters of formula R¹—(C═O)OR², R¹—(C═o)—R³—(C═O)R³ and R²O(C═o)—R³—COOR², wherein R¹ represents a C₈- to C₂₂-alkyl group, R² represents a C₃- to C₂₂-alkyl group and R³ represents a C₂- to C₁₆-alkylene group. The waxes or waxy substances have a solidification point above 40° C., preferably above 55° C. The needle penetration number (0.1 mm, 100 g, 5 s, 25° C.; according to DIN 51 579) is preferably in a range of 2 to 70, especially preferably 3 to 40. Preferably it contains at least one wax, which has a needle penetration number of less than 40, especially preferably less than 20. Carnauba wax and ceresin with a needle penetration number of less than 20 and their mixtures are especially preferred.

The subject matter of the invention also includes a process for manufacturing a hair or skin cleansing composition, in which the composition contains at least one wash-active surfactant and if necessary additional additives. In a preferred embodiment of the process at least one highly concentrated alkyl ether sulfate and an aqueous phase are fed separately to a micromixer, the alkyl ether sulfate is dissolved in the aqueous phase during passage through the micromixer and subsequently the remaining effective ingredient and additive substances are added. The term “aqueous phase” includes water and mixtures of water with soluble solvents, such as lower alcohols, e.g. ethanol or isopropanol, or polyols, such as ethylene glycol, diethylene glycol, butylene glycol or glycerol, preferably however water. The process can be performed batchwise or in a continuous variant. The hair or skin cleansing compositions include shampoos, shower baths, shower gels, bath preparations, etc. Usually highly concentrated, pasty 70% alkyl ether sulfates are used to make this sort of preparation. Their dilution has a special position in conventional manufacturing of these preparations. The dilution is especially difficult because of the viscosity behavior. A viscosity minimum is present in the concentrated range of 65 to 75%. The viscosity passes through a maximum in a concentration range of 30 to 60% and the alkyl ether sulfate is present as an extremely highly viscous stiff gel there. At concentrations under 30% (in regard to aqueous dilution) it is present in a low viscosity, sufficiently workable form. Even using dynamic high performance mixing when concentrated alkyl ether sulfate is introduced into water gel-like knots form and gelation occurs, which prevent an immediate and complete dissolution, and in general subsequently dissolve slowly. There is an undesirable after-thickening of the finished product as an undesirable consequence. That is the final viscosity and consistency are only reach after a certain storage time. In order to avoid these viscosity-dependent difficulties the product is circulated through a homogenizer for a comparatively long time, in order to achieve as complete dissolution as possible, which is very time consuming. Furthermore an electrolyte, such as sodium chloride, is added to the solution. This causes a clear viscosity reduction, an improved processability and more rapid dissolution in the concentrated region, in which alkyl ether sulfate is normally a rigid gel (30 to 60%). However in the resulting diluted alkyl ether sulfate solution sodium chloride generally has the opposite effect, namely a viscosity increasing effect. This has the disadvantages that more time is required for subsequent degassing for the viscous solution during manufacture, that further processing, e.g. the subsequent introduction of additional effective and auxiliary ingredients is more difficult and time intensive and that after-thickening cannot be completely excluded.

Suitable alkyl ether sulfates have alkyl groups with 8 to 22, preferably 10 to 16, carbon atoms and an ethoxylation degree of 1 to 20, preferably 1 to 4. Lauryl ether sulfate is especially preferred. Alkali metal or alkaline earth metal cations, e.g. sodium ions, magnesium ions or also ammonium ions, are suitable counter ions. Suitable alkyl ether sulfates are described, e.g., in “International Cosmetic Ingredient Dictionary and Handbook”, 7th Edition, Volume 2 in the surfactant section, particularly under ‘Alkyl Ether Sulfates’. In a preferred embodiment the highly concentrated alkyl ether sulfates are dissolved without addition of electrolytes, especially without addition of NaCl. When the end product is a viscose composition, the desired final viscosity is preferably adjusted at the conclusion of the manufacturing process by addition of an electrolyte, such as NaCl, or another conventional viscosity-increasing substance. For this purpose thickeners are used, such as guar gum, silicates, methyl cellulose, hydroxyethyl cellulose or carboxy vinyl polymers. Co-surfactants can also be used as thickeners.

In the process according to the invention highly concentrated alkyl ether sulfate is dissolved by means of a micromixer, preferably in electrolyte-free water. Subsequently the effective ingredients and additive ingredients are stirred in. The following ingredients come under consideration as effective ingredients and additive ingredients: additional anionic, nonionic or amphoteric surfactants, anti-flaking agents, hair and skin care ingredients, like quaternary alkylamines, cationic polymers of natural or synthetic origin, proteins and their derivatives, such as collagen protein hydrolyzate, keratin protein hydrolyzate, silk protein hydrolyzate, wheat protein hydrolyzate and silicone compounds. In addition, the following can be used: perfume oils, dyestuffs, turbidity-inducing agents, such as e.g. glycol distearate; hair-conditioning agents, such as synthetic or natural phopholipids or quaternary derivatives of starch or cellulose; solvating agents, such as short-chain alcohols, e.g. ethanol, n-propanol, isopropanol or glycols, such as butylene or propylene glycol; amino acids, such as histidine, glycine, alanine, threonine, arginine, cysteine and their derivatives, e.g. fatty acid condensation products or quaternary products; additional effective ingredients, such as plant extracts, vitamins, allantoin, chitosan, preservatives; etc.

The subject matter of the invention also includes a process for manufacturing of cosmetic sun-protecting or sunscreen preparations, in which the preparations contain at least one light protective agent and the preparation or at least one part of the preparation is conducted through an apparatus with microstructured units. Disperse sunscreen compositions, which either contain insoluble light protecting agents in finely dispersed form or dispersed sun-protecting agents, and which comprise an oil phase or a lipid phase and an aqueous phase. They can be O/W or W/O emulsions. The light protective ingredient can be UV absorbing inorganic pigments, inorganic nanopigments and oil or water soluble organic UVA-, UVB- or UVA/UVB filtering substances. Suitable filtering substances include, e.g., 2-phenylbenzimidazol-5-sulphonic acid and its salts, cinnammic acid derivatives, salicylic acid derivatives, camphor derivatives, triazine derivatives, benzophenone derivatives, dibenzoylmethane derivatives, β,β-diphenylacrylate derivatives, p-aminobenzoic acid derivatives, menthylanthranilates, polymers with light-protecting action and silicones with light-protecting action. The light-protecting agents according to the invention are characterized by an improved sun protection factor.

The subject matter of the invention also includes a process for manufacturing hair care compositions comprising a hydrophilic phase and a hydrophobic phase and containing at least one effective ingredient, which is a C₁₀- to C₃₀-fatty alcohol and/or a cationic surfactant. Preferably the hair care composition is a fatty alcohol dispersion, in which the fatty alcohol is present as a solid at room temperature (25° C.) and is dispersed in a melted state in an aqueous phase by means of a micromixer. The fatty alcohol can be present in an amount of 0.1 to 20 percent by weight, preferably 0.5 to 10 percent by weight, especially preferably from 1 to 8 percent by weight. Primary alcohols are suitable fatty alcohols, especially 1-alkanols with 6 to 26 carbon atoms, preferably 12 to 22 carbon atoms. The use of octanol, decanol, dodecanol, lauryl alcohol, tetradecanol, myrisitic alcohol, hexadecanol, cetyl alcohol, octadecanol, stearyl alcohol, mixtures of these fatty alcohols, has proven to be advantageous. Cetyl alcohol is an especially preferred fatty alcohol.

The cationic surfactants in the hair care compositions are preferably contained in an amount of 0.01 to 10, especially preferably of 0.05 to 5 percent by weight. Suitable cationic surfactants are surfactants, which contain a quaternary ammonium group and can be represented by the general formula (I),

N⁽⁺⁾R¹R²R³R⁴X⁽⁻⁾ (1),

wherein R¹ to R⁴, independently of each other, represent aliphatic groups, aromatic groups, alkoxy groups, polyoxyalkylene groups, alkylamido groups, hydroxyalkyl groups, aryl groups or alkaryl groups with 1 to 22 carbon atoms and X⁻ represents an anion, e.g. a halogen, acetate, phosphate, nitrate or alkyl sulfate, preferably a chloride. The aliphatic groups can also contain cross-links or other groups, for example additional amino groups. For example, the chlorides or bromides of alkyldimethylbenzyl ammonium salts, alkyltrimethyl ammonium salts, e.g. cetyltrimethyl ammonium chloride or -bromide, tetradecyltrimethyl ammonium chloride or -bromide, alkyldimethylhydroxyethyl ammonium chloride or -bromide, alkylpyridinium salts, e.g. lauryl- or cetylpyridinium chloride, alkylamidoethyltrimethyl ammonium ether sulfate and compounds with cationic character, such as amine oxides, e.g. alkylmethyl amine oxides or alkylaminoethyldimethyl amine oxides, are, for example, suitable cationic surfactants. Cetyltrimethylammonium chloride is particularly preferred. The hair care composition can contain at least one co-emulsifying cationic polymer, e.g. vinylimidazolium methochloride/vinylpyrrolidone copolymer (Polyquaternium-16), quaternized vinylpyrrolidone/dimethylaminoethylmethacrylate copolymer (Polyquaternium-11), cationic silicone polymers, e.g. diquaternary polydimethyl siloxane (Quaternium-80), polyquaternium-10 or cationic guar derivatives, such as guar hydroxypropyltrimonium chloride or mixtures of the foregoing compounds.

The hair care composition is produced, when at least one fatty alcohol is melted with suitable heating, especially between 60° C. and 100° C., preferably between 70° C. and 90° C. and an emulsifier, which preferably is nonionic, is added in an amount of 0.5 to 20 percent by weight, especially preferably from 1.0 to 10.0 percent by weight. The oil phase is dispersed with an aqueous phase containing the cationic surfactant by means of a micromixer. The cooling occurs preferably by means of a microheat exchanger. The finished hair care preparation can be directly filled in a final packaging unit. In one embodiment it is a matter of a creamy, highly viscous hair care composition, which preferably is rinsed out after application (rinse product). The fatty alcohol content amounts preferably to 0.01 to 20 percent by weight, especially preferably from 1 to 10 percent by weight. The viscosity preferably amounts to from 1000 to 10000, especially preferably from 1500 to 8000 mPa s, measured as a dynamic viscosity measurement with a HAAKE rotation viscometer VT550 at a temperature of 25 C with a test body according to DIN 53019 (SV-DIN) and a shear rate of 50 s⁻¹.

The subject matter of the invention also includes a process for manufacturing leave-in hair care compositions. They comprise a hydrophilic and a hydrophobic phase, which are dispersed by means of a micromixer. They contain substantially the same ingredients as in the above-mentioned hair care compositions. The content of the hydrophobic phase is reduced in contrast to creamy, rinse-out hair care compositions, so that no viscous or fluid crystalline structures form. The viscosity is clearly reduced and the products are sprayable with conventional mechanical spray apparatus, e.g. mechanically operated spray pumps. The fatty alcohol content of the leave-in product amounts to preferably from 0.01 to 3, especially preferably from 0.1 to 1, percent by weight. The viscosity of the leave-in products preferably amounts to 100 to 2000, especially 300 to 1500 mPa s, measured as a dynamic viscosity measurement with a HAAKE rotation viscometer VT550 at a temperature of 25° C. with a test body according to DIN 53019 (SV-DIN) and a shear rate of 50 s⁻¹. The sprayability of the leave-in hair care compositions made by the methods of the invention is clearly improved in contrast to the conventionally made sprayable hair care compositions.

The subject matter of the invention also is a process for manufacturing hair dye compositions, which contain at least one hair coloring substance or at least one oxidation dye pre-cursor product. In this process the preparation or at least a part of the preparation is conducted through an apparatus with microstructured units. The hair coloring substance can be a hair coloring pigment or an organic direct dye compound taken up directly by the hair.

The production of hair dye creams according to the invention, especially for oxidation dye pre-cursor products, is especially preferred. These hair dye creams are usually based on a wax-containing cream. In conventional manufacturing processes a heated hydrophobic wax-phase melted at about 70 to 80° C. is emulsified at a temperature of about 70 to 80° C. in an aqueous phase containing the dye pre-cursor product and if necessary additional additive ingredients. Subsequently they must be very slowly cooled and stirred, in order to prevent uncontrolled re-crystallization and precipitation of the wax (seediness). Undesirable reactions can occur during this comparatively long duration cooling because of stirring in of oxygen. Furthermore an after-thickening can occur, which produce difficulties during filling of the finished product. When a microheat exchanger is used for cooling, the danger of seediness or pepperiness and undesirable oxidation reactions is reduced. The contained dye mass can be filled immediately as a finished product. A substantially finer distribution of the hydrophobic phase, a substantially greater specific surface area and thus a higher effectiveness of the raw materials are achieved by dispersing with a micromixer. This has the result that a substantially smaller amount of raw materials can be used and the coloring or dyeing properties can be improved. The temperature of the aqueous phase can be selected so that the mixing temperature resulting after emulsifying the wax melt is below the solidification temperature of the wax. The dye cream can be filled immediately after that without further cooling.

The preferred hair dye composition contains (a) water, (b) at least one waxy or fatty substance at room temperature (25° C.), (c) at least one surfactant and (d) at least one direct hair dye compound or at least one oxidation dye precursor. The total amount of dyestuffs or dye precursor compounds in the composition of the invention is preferably about 0.01 to 10 percent by weight, especially preferably from about 0.2 to 7 percent by weight. Suitable direct-dyeing dye compounds include e.g. triphenylmethane dye compounds, aromatic nitro dye compounds, azo dye compounds, quinone dye compounds, cationic or anionic dye compounds. The following compounds are suitable:

Nitro Dye Compounds (Blue):

1,4-bis[(2-hydroxyethyl)amino]-2-nitrobenzene, 1-(2-Hydroxyethyl)amino-2-nitro-4-[di(2-hydroxyethyl)amino]-benzene (HC Blue No. 2), 1-Amino-3-methyl-4-[(2-hydroxyethyl)amino]-6-nitrobenzene (HC Violet No. 1), 4-[Ethyl-(2-hydroxyethyl)-amino]-1-[(2-hydroxyethyl)amino]-2-nitrobenzene hydrochloride (HC Blue No. 12), 4-[Di(2-hydroxyethyl)amino]-1-[(2-methoxyethyl)amino]-2-nitrobenzene (HC Blue No. 11), 1-[(2,3-Dihydroxypropyl)amino]-4-[methyl-(2-hydroxyethyl)amino]-2-nitrobenzene (HC Blue No. 10), 1-[(2,3-Dihydroxypropyl)-amino]-4-[ethyl-(2-hydroxyethyl)amino]-2-nitrobenzene hydrochloride (HC Blue No. 9), 1-(3-Hydroxypropylamino)-4-[di(2-hydroxyethyl)amino]-2-nitrobenzene (HC Violet No. 2), 1-Methylamino-4-[methyl-(2,3-dihydroxypropyl)amino]-2-nitrobenzene (HC Blue No. 6), 2-((4-Amino-2-nitrophenyl)amino)-5-dimethylaminobenzoic acid (HC Blue No. 13), 1-(2-Aminoethylamino)-4-[di(2-hydroxyethyl)amino]-2-nitrobenzene, 4-(Di(2-hydroxyethyl)amino)-2-nitro-1-phenylaminobenzene.

Nitro Dye Compounds (Red):

1-Amino-4-[(2-hydroxyethyl)amino]-2-nitrobenzene (HC Red No. 7), 2-Amino-4,6-dinitrophenol, 1,4-Diamino-2-nitrobenzene (CI76070), 4-Amino-2-nitrodiphenylamine (HC Red No. 1), 1-Amino-4-[di(2-hydroxyethyl)amino]-2-nitro-benzene hydrochloride (HC Red No. 13), 1-Amino-5-chloro-4-[(2-hydroxyethyl)-amino]-2-nitrobenzene, 4-Amino-1-[(2-hydroxyethyl)amino]-2-nitrobenzene (HC Red No. 3), 4-((2-Hydroxyethyl)methylamino)-1-(methylamino)-2-nitrobenzene, 1-Amino-4-((2,3-dihydroxypropyl)amino)-5-methyl-2-nitrobenzene, 1-Amino-4-(methylamino)-2-nitrobenzene, 4-Amino-2-nitro-1-((prop-2-en-1-yl)-amino)benzene, 4-Amino-3-nitrophenol, 4-[(2-Hydroxyethyl)-amino]-3-nitrophenol, 4-[(2-Nitrophenyl)amino]phenol (HC Orange No. 1), 1-[(2-Aminoethyl)-amino]-4-(2-hydroxyethoxy)-2-nitrobenzene (HC Orange No. 2), 4-(2,3-Dihydroxypropoxy)-1-[(2-hydroxyethyl)amino]-2-nitrobenzene (HC Orange No. 3), 1-Amino-5-chloro-4-[(2,3-dihydroxypropyl)amino]-2-nitrobenzene (HC Red No. 10), 5-Chlor-1,4-[di(2,3-dihydroxypropyl)amino]-2-nitrobenzene (HC Red No. 11), 2-[(2-Hydroxyethyl)-amino]-4,6-dinitrophenol, 4-Ethylamino-3-nitrobenzoic acid, 2-[(4-Amino-2-nitrophenyl)amino]benzoic acid, 2-Chlor-6-ethylamino-4-nitrophenol, 2-Amino-6-chloro-4-nitrophenol, 4-[(3-Hydroxypropyl)amino]-3-nitrophenol, 2,5-Diamino-6-nitropyridine, 6-Amino-3-((2-hydroxyethyl)amino)-2-nitropyridine, 3-Amino-6-((2-hydroxyethyl)amino)-2-nitropyridine, 3-Amino-6-(ethylamino)-2-nitropyridine, 3-((2-Hydroxyethyl)amino)-6-(methylamino)-2-nitropyridine, 3-Amino-6-(methylamino)-2-nitropyridine, 6-(Ethylamino)-3-((2-hydroxyethyl)amino)-2-nitropyridine, 1,2,3,4-Tetrahydro-6-nitroquinoxaline, 7-Amino-3,4-dihydro-6-nitro-2H-1,4-benzoxazine (HC Red No. 14).

Nitro Dye Compounds (Yellow):

1,2-Diamino-4-nitrobenzene (CI76020), 1-Amino-2-[(2-hydroxyethyl)amino]-5-nitrobenzene (HC Yellow No. 5), 1-(2-Hydroxy-ethoxy)-2-[(2-hydroxyethyl)-amino]-5-nitrobenzene (HC Yellow No. 4), 1-[(2-Hydroxyethyl)amino]-2-nitrobenzene (HC Yellow No. 2), 2-(Di(2-hydroxyethyl)amino)-5-nitrophenol, 2-[(2-Hydroxyethyl)-amino]-1-methoxy-5-nitrobenzene, 2-Amino-3-nitrophenol, 1-Amino-2-methyl-6-nitrobenzene, 1-(2-Hydroxyethoxy)-3-methylamino-4-nitrobenzene, 2,3-(Dihydroxypropoxy)-3-methylamino-4-nitrobenzene, 2-[(2-Hydroxyethyl)amino]-5-nitrophenol (HC Yellow No. 11), 3-[(2-Aminoethyl)amino]-1-methoxy-4-nitrobenzene hydrochloride (HC Yellow No.9), 1-[(2-Ureidoethyl)-amino]-4-nitrobenzene, 4-[(2,3-Dihydroxypropyl)amino]-3-nitro-1-trifluormethylbenzene (HC Yellow No. 6), 1-Chloro-2,4-bis[(2-hydroxyethyl)amino]-5-nitrobenzene (HC Yellow No. 10), 1-Amino-4-((2-aminoethyl)amino)-5-methyl-2-nitrobenzene, 4-[(2-Hydroxyethyl)-amino]-3-nitro-1-methylbenzene, 1-Chlor-4-[(2-hydroxyethyl)amino]-3-nitrobenzene (HC Yellow No. 12), 4-[(2-hydroxyethyl)amino]-3-nitro-1-trifluormethylbenzene (HC Yellow No. 13), 4-[(2-Hydroxyethyl)amino]-3-nitrobenzonitrile (HC Yellow No. 14), 4-[(2-Hydroxyethyl)amino]-3-nitro-benzamid (HC Yellow No. 15), 3-((2-Hydroxyethyl)amino)-4-methyl-1-nitrobenzene, 4-Chlor-3-((2-hydroxyethyl)-amino)-1-nitrobenzene.

Quinone Dye Compounds:

1,4-Di[(2,3-dihydroxypropyl)amino]-9,10-anthraquinone, 1,4-Di[(2-hydroxyethyl)-amino]-9,10-anthraquinone (CI61545 Disperse Blue 23), 1-[(2-Hydroxyethyl)-amino]-4-methylamino-9,10-anthraquinone (CI61505, Disperse Blue No. 3), 2-[(2-Aminoethyl)amino]-9,10-anthraquinone (HC Orange No. 5), 1-Amino-4-hydroxy-9,10-anthraquinone (CI60710, Disperse Red 15), 0.1-Hydroxy-4-[(4-methyl-2-sulfophenyl)amino]-9,10-anthraquinone, 7-Beta-D-glucopyranosyl-9,10-dihydro-1-methyl-9,10-dioxo-3,5,6,8-tetrahydroxy-2-anthracencarboxylic acid (CI75470, Natural Red 4), 1-[(3-Aminopropyl)amino]-4-methylamino-9,10-anthraquinone (HC Blue No. 8), 1-[(3-Aminopropyl)amino]-9,10-anthraquinone (HC Red No. 8), 1,4-Diamino-2-methoxy-9,10-anthraquinone (CI62015, Disperse Red No. 11, Solvent Violet No. 26), 1,4-Dihydroxy-5,8-bis[(2-hydroxyethyl)-amino]-9,10-anthraquinone (CI62500, Disperse Blue No. 7, Solvent Blue No. 69), 1,4-Diamino-9,10-anthraquinone (CI61100, Disperse Violet No. 1), 1-Amino-4-(methylamino)-9,10-anthraquinone (CI61105, Disperse Violet No. 4, Solvent Violet No. 12), 2-Hydroxy-3-methoxy-1,4-naphthaquinone, 2,5-Dihydroxy-1,4-naphthaquinone, 2-Hydroxy-3-methyl-1,4-naphthaquinone, N-(6-((3-Chlor-4-(methylamino)phenyl)imino)-4-methyl-3-oxo-1,4-cyclohexadien-1-yl)urea (HC Red No. 9), 2-((4-(Di(2-hydroxyethyl)amino)phenyl)amino)-5-((2-hydroxyethyl)-amino)-2,5-cyclohexadien-1,4-dione (HC Green No. 1), 5-Hydroxy-1,4-naphthaquinone (CI75500, Natural Brown No. 7), 2-Hydroxy-1,4-naphthoquinone (CI75480, Natural Orange No. 6), 1,2-Dihydro-2-(1,3-dihydro-3-oxo-2H-indol-2-yliden)-3H-indol-3-one (CI73000), 4-((5-((2-Hydroxyethyl)amino-1-methyl-1H-pyrazol-4-yl)imino)-4 ,5-dihydro-5-((2-hydroxyethyl)-imino)-1-methyl-1H-pyrazole sulfate (1:1), hydrate (1:1).

Basic Dye Compounds:

9-(Dimethylamino)-benzo[a]phenoxazin-7-ium chloride (CI51175; Basic Blue No.6), Di[4-(diethylamino)phenyl][4-(ethylamino)naphthyl]carbenium chloride (CI42595; Basic Blue No. 7), Di-(4-(dimethylamino)phenyl)-(4-(methylphenylamino)-napthalene-1-yl)carbenium chloride (CI42563; Basic Blue No. 8), 3,7-Di(dimethylamino)phenothiazin-5-ium chloride (CI52015 Basic Blue No. 9), Di[4-(dimethylamino)phenyl][4-(phenylamino)naphthyl]carbenium chloride (CI44045; Basic Blue No.26), 2-[(4-(Ethyl(2-hydroxyethyl)amino)phenyl)azo]-6-methoxy-3-methyl-benzothiazolium methyl sulfate (CI11154; Basic Blue No. 41), 8-Amino-2-bromo-5-hydroxy-4-imino-6-[(3-(trimethylammonio)-phenyl)-amino]-1 (4H)-napthalinone chloride (CI56059; Basic Blue No. 99), bis-[4-(dimethylamino)phenyl]-[4-(methylamino)phenyl]carbenium chloride (CI42535; Basic Violet No. 1), Tri(4-amino-3-methylphenyl)carbenium chloride (CI42520; Basic Violet No. 2), Tris[4-(dimethylamino)-phenyl]carbenium chloride (CI42555; Basic Violet No. 3), 2-[3,6-(Diethylamino)dibenzopyranium-9-yl]-benzoic acid chloride (CI45170; Basic Violet No. 10), Di(4-aminophenyl)(4-amino-3-methylphenyl)carbenium chloride (CI42510 Basic Violet No. 14), 1,3-bis[(2,4-diamino-5-methylphenyl)azo]-3-methylbenzene (CI21010; Basic Brown No. 4), 1-[(4-Aminophenyl)azo]-7-(trimethylammonio)-2-naphthol chloride (CI12250; Basic Brown No. 16), 3-[(4-Amino-2,5-dimethoxyphenyl)azo]-N,N,N-trimethylbenzene aminium chloride (CI12605, Basic Orange No. 69), 1-[(4-Amino-2-nitrophenyl)az6]-7-(trimethylammonio)-2-naphthol chloride, 1-[(4-Amino-3-nitrophenyl)azo]-7-(trimethylammonio)-2-naphthol chloride (CI2251; Basic Brown No. 17), 3,7-Diamino-2,8-dimethyl-5-phenylphenazinium chloride (CI50240; Basic Red No. 2), 1,4-Dimethyl-5-[(4-(dimethylamino)phenyl)azo]-1,2,4-triazolium chloride (CI1055; Basic Red No. 22), 2-Hydroxy-1-[(2-methoxyphenyl)azo]-7-(trimethylammonio)-napthalene chloride (CI12245; Basic Red No. 76), 2-[2-((2,4-Dimethoxyphenyl)amino)ethenyl]-1,3,3-trimethyl-3H-indolium chloride (CI48055; Basic Yellow No. 11), 3-Methyl-1-phenyl-4-[(3-(trimethylammonio)-phenyl)azol-pyrazo]-5-one chloride (CI12719; Basic Yellow No. 57), Di[4-(dimethylamino)phenyl]-iminomethane hydrochloride (CI41000; Basic Yellow No. 2), bis-[4-(diethylamino)-phenyl]phenyl carbenium hydrogen sulfate (1:1) (CI42040; Basic Green No. 1), Di(4-(dimethylamino)phenyl)phenylmethanol (CI42000; Basic Green No. 4), 1-(2-Morpholinium-propylamino)-4-hydroxy-9,10-anthraquinone methyl sulfate, 1-((3-(Dimethylpropylaminium)propyl)amino]-4-(methylamino)-9,10-anthraquinone chloride.

Neutral Azo Dye Compounds:

1-[Di(2-hydroxyethyl)amino]-3-methyl-4-[(4-nitrophenyl)azo]-benzene (CI11210, Disperse Red No. 17), 1-[Di(2-hydroxyethyl)amino]-4-[(4-nitrophenyl)azo]-benzene (Disperse Black No. 9), 4-[(4-Aminophenyl)azo]-1-[di(2-hydroxyethyl)-amino]-3-methylbenzene (HC Yellow No. 7), 2,6-Diamino-3-[(pyridine-3-yl)azo]-pyridine, 2-((4-(Acetylamino)phenyl)azo)-4-methylphenol (CI1855; Disperse Yellow No. 3).

Acid Dye Compounds:

6-Hydroxy-5-[(4-sulfophenyl)azo]-2-napthalene sulfonic acid disodium salt (CI15985; Food Yellow No. 3; FD&C Yellow No. 6), 2,4-Dinitro-1-naphthol-7-sulfonic acid disodium salt (CI10316; Acid Yellow No. 1; Food Yellow No. 1), 2-(Indan-1,3-dion-2-yl)quinolin-x,x-sulfonic acid (mixture of mono and disulfonic acids) (CI47005; D&C Yellow No. 10; Food Yellow No. 13; Acid Yellow No. 3), 5-Hydroxy-1-(4-sulfophenyl)-4-[(4-sulfophenyl)azo]pyrazol-3-carboxylic acid trisodium salt (CI19140; Food Yellow No. 4; Acid Yellow No. 23), 9-(2-Carboxyphenyl)-6-hydroxy-3H-xanthen-3-one (CI45350; Acid Yellow No. 73; D&C Yellow No. 8), 4-((4-Amino-3-sulfophenyl)azo)benzenesulfonic acid disodium salt (CI13015, Acid Yellow No. 9), 5-[(2,4-Dinitrophenyl)amino]-2-phenylamino-benzene sulfonic acid sodium salt (CI0385; Acid Orange No. 3), 4-[(2,4-Dihydroxyphenyl)azo] benzene sulfonic acid monosodium salt (CI14270; Acid Orange No. 6), 4-[(2-Hydroxynaphth-1-yl)azo]-benzene sulfonic acid sodium salt (CI15510; Acid Orange No. 7), 4-[(2,4-Dihydroxy-3-[(2,4-dimethylphenyl)azo]-phenyl)azo]-benzene sulfonic acid sodium salt (CI20170; Acid Orange No. 24), 4-Hydroxy-3-[(4-sulfonaphth-1-yl)azo]-1 l-napthalene sulfonic acid disodium salt (CI14720; Acid Red No. 14), 4-Hydroxy-3-[(2-methoxyphenyl)azo]-1-napthalene sulfonic acid monosodium salt (CI14710; Acid Red No. 4), 6-Hydroxy-5-[(4-sulfonaphth-1-yl)azol-2,4-napthalene-disulfonic acid trisodium salt (Cl 16255; Ponceau 4R; Acid Red No. 18), 3-Hydroxy-4-[(4-sulfonaphth-1-yl)azo]-2,7-napthalene-disulfonic acid trisodium salt (CI16185; Acid Red No. 27), 8-Amino-1-hydroxy-2-(phenyl-azo)-3,6-napthalene-disulfonic acid disodium salt (CI7200; Acid Red No. 33), 5-(acetylamino)-4-hydroxy-3-[(2-methylphenyl)azo]-2,7-napthalene disulfonic acid disodium salt (CI18065; Acid Red No. 35), 2-(3-Hydroxy-2,4,5,7-tetraiodo-dibenzopyran-6-on-9-yl)-benzoic acid disodium salt (CI45430; Acid Red No. 51), N-[6-(Diethylamino)-9-(2,4-disulfophenyl)-3H-xanthen-3-yliden]-N-ethyl ethane ammonium hydroxide, inner salt, sodium salt (CI45100; Acid Red No. 52), 8-[(4-(Phenylazo)phenyl)azo]-7-naphthol-1,3-disulfonic acid disodium salt (CI27290; Acid Red No. 73), 2′,4′,5′,7′-Tetrabrom-3′,6′-dihydroxyspiro[isobenzofuran-1 (3H),9′-[9H]xanthen]-3-one disodium salt (CI45380 Acid Red No. 87), 2′,4′,5′,7′-Tetrabromo-4,5,6,7-tetrachloro -3′,6′-dihydroxyspiro-[isobenzofuran-1 (3H),9′[9H]xanthen]-3-one disodium salt (CI45410; Acid Red No. 92), 3′,6′-Dihydroxy-4′,5′-diiodospiro[isobenzofuran-1 (3H),91 (9H)-xanthen]-3-one disodium salt (CI45425; Acid Red No. 95), 2-Hydroxy-3-((2-hydroxy-naphth-1-yl)azo)-5-nitrobenzene sulfonic acid monosodium salt (CI15685; Acid Red No. 184), (2-Sulfophenyl)-di[4-(ethyl((4-sulfophenyl)methyl)amino)phenyl]-carbenium, disodium salt, betaine (CI42090; Acid Blue No. 9; FD&C Blue No. 1), 1,4-bis-[(2-sulfo-4-methylphenyl)amino]-9, 10-anthraquinone-disodium salt (CI61570; Acid Green No. 25), bis[4-(dimethylamino)phenyl]-(3,7-disulfo-2-hydroxynaphth-1-yl)carbenium inner salt, monosodium salt (CI44090; Food Green No. 4; Acid Green No. 50), bis-[4-(diethylamino)-phenyl](2,4-disulfophenyl)carbenium inner salt, sodium salt (2:1) (CI42045; Food Blue No. 3; Acid Blue No. 1), bis[4-(diethylamino)phenyl](5-hydroxy-2,4-disulfophenyl)-carbenium inner salt, calcium salt (2:1) (CI42051; Acid Blue No. 3), 1-Amino-4-(cyclohexylamino)-9,10-anthraquinone-2-sulfonsdure sodium salt (CI62045; Acid Blue No. 62), 1-Amino-4-(phenylamino)-9,10-anthraquinone-2-sulfonic acid (CI62055; Acid Blue No. 25), 2-(1,3-Dihydro-3-oxo-5-sulfo-2H-indol-2-yliden)-2,3-dihydro-3-oxo-1H-indol-5-sulfonic acid disodium salt (CI73015; Acid Blue No. 74), 9-(2-carboxyphenyl)-3-[(2-methylphenyl)amino]-6-[(2-methyl-4-sulfophenyl)-amino]xanathylium inner salt, monosodium salt (CI45190; Acid Violet No. 9), 1-Hydroxy-4-[(4-methyl-2-sulfophenyl)amino]-9,10-anthraquinone-sodium salt (CI60730; D&C Violet No. 2; Acid Violet No. 43), bis[3-nitro-4-[(4-phenylamino)-3-sulfophenylamino]-phenyl]sulfone (CI10410; Acid Brown No. 13), 5-Amino-4-hydroxy-6-[(4-nitrophenyl)-azo]-3-(phenylazo)-2,7-napthalene disulfonic acid disodium salt (CI20470; Acid Black No. 1), 3-Hydroxy-4-[(2-hydroxynaphth-1-yl)azo]-7-nitro-1-napthalene-sulfonic acid chromium complex (3:2) (CI15711; Acid Black No. 52), 3-[(2,4-Dimethyl-5-sulfophenyl)azo]-4-hydroxy-1-napthalene sulfonic acid disodium salt (CI4700; Food Red No. 1; Ponceau SX; FD&C Red No. 4), 4-(Acetylamino)-5-hydroxy-6-[(7-sulfo-4-[(4-sulfophenyl)azo]naphth-1-yl)azo]-1,7-napthalene disulfonic acid tetrasodium salt (CI28440; Food Black No. 1), 3-Hydroxy-4-(3-methyl-5-oxo-1-phenyl-4 ,5-dihydro-1H-pyrazol-4-yl-azo)- napthalene-1-sulfonic acid sodium salt, chromium complex (Acid Red No. 195).

E. Sagarin, “Cosmetics, Science and Technology”, Interscience Publishers Inc., New York (1957), pp. 503 ff.; H. Janistyn, “Handbook of Cosmetics and Fragrances”, Vol 3 (1973), pp. 388 ff. and K. Schrader “Foundations and Formulations of Cosmetics”, 2^{nd} Ed. (1989), pp. 782 to 815 describe additional or further known and conventional hair dye compounds for dyeing hair, which can be contained in the hair dye compositions according to the invention.

Suitable hair dying pigments are coloring agents, which are practically insoluble in the application medium, and can be inorganic or organic. Also inorganic-organic mixed pigments are possible. The pigments however are preferably not nanopigments. The preferred particle size amounts to 1 to 200 μm, especially 3 to 150 μm, and especially preferably from 10 to 100 μm. Inorganic pigments are preferred. The inorganic pigments can be of a natural origin, for example chalk, ocher, umber, green earth, burnt Terra di Siena or graphite. The pigments can be white pigments, such as titanium dioxide or zinc oxide, black pigments, such as iron oxide black, colored pigments, such as ultramarine or iron oxide red, lustrous pigments, metal effect pigments, pearlescent pigments and fluorescence or phosphorescence pigments. Preferably at least one pigment is a non-white pigment. Metal oxides, metal hydroxides and metal oxyhydrates, mixed phase pigments, sulfur-containing silicates, metal sulfides, complex metal cyanides, metal sulfates, metal chromates and metal molybdates, as well as metals themselves (Bronze pigments) are suitable. Especially titanium dioxide (CI 77891), black iron oxide (CI 77499), yellow iron oxide (CI 77492), red and brown iron oxide (CI 77491), manganese violet (CI 77742), Ultramarine (Sodium aluminum sulfosilicate, CI 77007, Pigment Blue 29), Chromium oxide hydrate (CI 77289), iron blue (Ferric ferrocyanide, CI 77510), Carmine (Cochineal), are suitable as the pigments in embodiments of the cosmetic preparations according to the invention. Pigments based on mica, which are coated with a metal oxide or a metal oxychloride, such as titanium dioxide or bismuth oxychloride and, if necessary, other coloring agents, such as iron oxides, iron blue, Ultramarine, Carmine, etc, are particularly preferred. The colors of these particular preferred pigments are determined by variation of the coating thickness. These pigments are marketed by Merck, Germany, under the trademarks RONA®, COLORONA®, DICHRONA® and TIMIRON®. Organic pigments include, e.g., the natural pigments sepia, gamboge, animal charcoal, Cassel brown, indigo, chlorophyll and other plant pigments. Synthetic organic pigments are, e.g., azo pigments, anthraquinoid, indigoid, dioxazine, quinacridine, phthalocyanine, isoindolinone, perylene and perinone, metal complex, alkali blue and diketo-pyrrolopyrrole pigments.

An additional embodiment of the process according to the invention is a process for manufacturing cosmetic skin cream, in which the product is an emulsion formed from a hydrophobic phase and an aqueous phase, contains at least one skin care active ingredient and the phases are dispersed in a micromixer. The skin cream usually contains water, a fatty or waxy substance, an emulsifier and a skin care ingredient. The active ingredient can be a cosmetic oil, emollient, vitamin, vitamin derivative, provitamin, essential fatty acid, sphingolipid, phospholipid, ceramide, betaine, panthenol. The skin cream according to the invention is characterized by an improved skin feel, an improved distributability of the effective ingredient, an improved delivery of the effective ingredient into the skin and a reduction of the required amount used.

An additional embodiment of the process according to the invention is a process for manufacturing of hair cosmetic or skin cosmetic preparations, which contain at least one powdery solid in a finely dispersed form, in which the dispersion of the solid is performed in a micromixer. Suitable solids include, e.g. pigments, pearlescence-imparting agents, talcum powder, mica, kaolin, zinc oxide, titanium oxide, precipitated calcium carbonate, magnesium carbonate or hydrogen carbonate, silicic acid, glass balls, ceramic balls or powdery polymeric material.

An additional embodiment of the process according to the invention is a process for manufacturing of hair cosmetic or skin cosmetic preparations, which contain at least one preservative, in which distribution of the preservative occurs in the micromixer. The preparation requires less preservative than current conventional preparations and has improved skin compatibility.

The advantages of the skin and hair cosmetic products manufactured according to the invention include an optimum particle distribution for particles homogenized into the product, an optimum distribution of the disperse phase in the external phase, a high effective surface area, a reduced need for emulsifier and, because of that, improved skin compatibility, improved effectiveness of effective ingredients and auxiliary substances, improved crystallization behavior and improved rheological properties. The hair treatment compositions made according to the invention have the advantage that they provide a more uniform deposition of the effective ingredients on the hair than conventional products. The narrower particle size distribution improves the up-take of the effective ingredients by the hair.

The following examples illustrate the above-described invention in more detail, but the details in these examples should not be considered as limiting the claims appended hereinbelow.

### EXAMPLES

In the following examples the components were mixed by means of a micromixer according to example 1 of EP 0 861 121 B1.

Example 1: Hair Rinse

| | |
|---|---|
| 4.3 g | Cetearyl alcohol (LANETTE ® O) |
| 0.4 g | Cetyl lactate |
| 0.5 g | Petrolatum (VASELINE ®) |
| 1.2 g | Cetyltrimethyl ammonium chloride |
| 0.45 g | Polyvinyl pyrrolidone |
| to 100 g | Water |

Example 2: Hair Care Preparation

| | |
|---|---|
| 5.5 g | Cetearyl alcohol (LANETTE ® O) |
| 1.2 g | Petrolatum (VASELINE ®) |
| 1.0 g | Paraffinum Liquidum |
| 0.5 g | Dimethylpolysiloxane (BELSIL ® DM 500) |
| 0.3 g | Lanolin alcohol |
| 0.2 g | Lanolin |
| 1.2 g | Cetyltrimethyl ammonium chloride |
| 0.3 g | Citric acid |
| 0.4 g | Perfume |
| to 100 g | Water |

Example 3: Cream-form Hair Care Preparation

| | |
|---|---|
| 6 g | Cetearyl alcohol |
| 1.7 g | Glycerol |
| 1 g | Cetyltrimethyl ammonium chloride |
| 1 g | Plant oil |
| 0.5 g | Panthenol |
| 0.5 g | Silicon oil (Dimethylpolysiloxane) |
| 0.2 g | Perfume |
| to 100 g | Water |

Example 4: Leave-on Hair Care Spray Preparation

| | |
|---|---|
| 0.5 g | Cetyl alcohol |
| 0.3 g | Glycerol |
| 0.25 g | Cetyltrimethyl ammonium chloride |
| 0.2 g | Styrene/vinylpyrrolidone copolymer |
| 0.2 g | Panthenol |
| 0.2 g | Perfume |
| 4.8 g | Ethanol |
| to 100 g | Water |

Example 5: Oxidation Hair Dye Cream

| | |
|---|---|
| 17 g | Cetearyl alcohol |
| 1.9 g | Sodium lauryl sulfate |
| 1.4 g | Sodium lauryl ether sulfate |
| 2.1 g | Lanolin alcohol |
| 6.1 g | Glyceryl stearate |
| 0.4 g | Sodium cocoyl isoethionate |
| 1.4 g | Ammonia |
| 6 g | Isopropanol |
| 0.6 g | Sodium sulfite |
| 0.3 g | EDTA |
| 0.06 g | p-Aminophenol hydrochloride |
| 0.65 g | p-toluylenediamine sulfate |
| 0.26 g | resorcinol |
| 0.3 g | Perfume |
| to 100 g | Water |

Example 6: Hair Tinting Composition

| | |
|---|---|
| 4.5 g | Cetearyl alcohol |
| 1.7 g | Cetyltrimethyl ammonium chloride |
| 4.2 g | Isopropyl |
| 0.0025 g | Basic Violet 14 (C.I. Nr. 42510) |
| to 100 g | water |

Example 7: Hair Treatment Composition with volatile ingredients

| | |
|---|---|
| 10 g | Propylene glycol |
| 2 g | Cetearyl alcohol (LANETTE ® O) |
| 2 g | Cyclomethicone (Dow Corning 345 Fluid) |
| 0.4 g | Perfume |
| 0.2 g | PHB Methyl ester |
| 0.1 g | PHB Propyl ester |
| to 100 g | Water |

The aqueous phase, which is heated at 80° C., containing the propylene glycol and both PHB esters is mixed in a micromixer with the cetearyl alcohol melted at 80° C. The perfume and the volatile cyclomethicone are metered into the mixture between the micromixer and the microheat exchanger and the resulting mixture is cooled in the heat exchanger to room temperature.

Example 8: Hair Wax

| | |
|---|---|
| 10 g | Triceteareth-4 Phosphate |
| 6 g | PEG-200 Hydrogenated glyceryl palmate |
| 18 g | Caprylic/capric triglyceride |
| 0.5 g | Perfume |
| 0.5 g | Preservative |
| to 100 g | water |

The first three ingredients are melted at 80° C. and mixed with hot water, which contains a preservative, also at 80° C. The perfume is metered into the emulsion produced thereby between the micromixer and the microheat exchanger. The resulting perfumed emulsion is cooled immediately to 50° C. and filled directly into the marketed container or package.

Example 9: Shampoo

| | |
|---|---|
| 30 g | Sodium lauryl ether sulfate |
| 8 g | Cocamidopropylbetaine |
| 3 g | Glycol distearate |
| 0.5 g | perfume |
| 0.35 g | Sodium benzoate |
| 0.15 g | Sodium formiate |
| 0.2 g | Sodium chloride |
| to 100 g | Water |

Example 10: Anti-flaking Shampoo

| | |
|---|---|
| 30 g | Sodium lauryl ether sulfate |
| 8 g | Cocamidopropylbetaine |
| 3 g | Glycoldistearate |
| 1 g | Zinc pyrithione (ZINC OMADINE ®) |
| 0.5 g | Perfume |
| 0.35 g | Sodium benzoate |
| 0.15 g | Sodium formate |
| 0.2 g | Sodium chloride |
| to 100 g | Water |

Example 11: Creamy Shower Bath

| | | |
|---|---|---|
| 1. | 12 g | 70% sodium lauryl ether sulfate |
| 2. | to 100 g | Water |
| 3. | 0.7 g | Preservative |
| 4. | 0.4 g | Perfume |
| 5. | 7.5 g | Turbidity-inducing agent (20 wt. % PEG-3 |
| | | distearate and 18 wt. % sodium lauryl ether |
| | | sulfate in water) |
| 6. | 0.3 g | Polyquaternium-10 (cationic cellulose) |
| 7. | 0.4 g | PEG-7 Glyceryl cocoate |
| 8. | 0.3 g | D-panthenol |
| 9. | 5 g | Cocamidopropylbetaine, 30% in water |
| 10. | 1 g | NaCl |

Variant 1:

A pre-mixture is made with ingredients 1 and 2 in a cold process with a micromixer. The remaining ingredients are then mixed into the composition in a conventional propeller stirrer device one after the other.

Variant 2:

A pre-mixture, which is cooled with a cross-flow microheat exchanger according to FIGS. 6a/6b of EP 0 391 895 B1, is made with ingredients 1 and 2 in a hot process with a micromixer. The dissipated heat is fed back to heat this cross-flow microheat exchanger connected to the micromixer. The remaining ingredients are then mixed one after the other in a conventional propeller stirrer device.

Example 12: Sunscreen Composition

| | INCI/EU | Wt. % |
|---|---|---|
| Oil Phase: | | |
| Parsol 1789 | Butyl methoxydibenzoylmethane | 0.30 |
| Neo Heliopan AV/OA | Octyl methoxycinnamate | 10.00 |
| Hostaphat KL 340 N | Trilaureth-4 Phosphate | 0.60 |
| Hostacerin DGI | Polyglyceryl-2 Sesquiisostearate | 0.70 |
| Phenoxetol | Phenoxyethanol | 1.00 |
| Cetiol 868 | Octyl stearate | 5.00 |
| Primal 352 | Mineral Oil | 5.00 |
| Abil Wax 9801D | Cetyl dimethicone | — |
| Water Phase: | | |
| Carbopol 2984 | Carbomer | 0.30 |
| Sodium hydroxide | Sodium hydroxide | 0.06 |
| Glycerol 86% | Glycerol | 5.00 |
| Water, demineralized | Aqua | To 100 |

Example 13: Sunscreen Composition

| | INCI/EU | Wt. % |
|---|---|---|
| Oil Phase: | | |
| Parsol 1789 | Butyl methoxydibenzoylmethane | 0.30 |
| Neo Heliopan AV/OA | Octyl methoxycinnamate | 10.00 |
| Hostaphat KL 340 N | Trilaureth-4 Phosphate | 0.60 |
| Hostacerin DGI | Polyglyceryl-2 Sesquiisostearate | 0.70 |
| Phenoxetol | Phenoxyethanol | 1.00 |
| Cetiol 868 | Octyl stearate | 9.00 |
| Primol 352 | Mineral Oil | — |
| Abil Wax 9801D | Cetyl dimethicone | 1.00- |
| Water Phase: | | |
| Carbopol 2984 | Carbomer | 0.30 |
| Sodium hydroxide | Sodium hydroxide | 0.06 |
| Glycerol 86% | Glycerol | 5.00 |
| Water, demineralized | Aqua | To 100 |

Example 14: Sunscreen Composition

| | INCI/EU | Wt. % |
|---|---|---|
| Oil Phase: | | |
| Parsol 1789 | Butyl methoxydibenzoylmethane | 1.50 |
| PHB-methyl ester | Methyl parabene | 0.20 |
| Neo Heliopan AV/OA | Octyl methoxycinnamate | 10.00 |
| Neo Heliopan Type 303 | Octocrylene | 10.00 |
| Finsolv TN | C12-15 Alkyl Benzoate | 2.50 |
| Eutanol G | Octyldodecanol | 10.00 |
| Antaron V 216 | PVP/Hexadecene Copolymer | 2.00 |
| Vitamin E Acetate | Tocopheryl acetate | 0.50 |
| Perfume | Perfume | 0.30 |
| Abil Wax 9801D | Cetyl dimethicone | 0.50 |
| Water Phase: | | |
| Carbopol 1382 | Acrylates/C10-30 Aryl Acrylate | 0.45 |
| | Crosspolymer | |
| Colorona Oriental Beige | Mica (and) CI 77891 (and) CI 77491 | 0.05 |
| 17231 | | |
| Glycerol 86% | Glycerol | 5.00 |
| Edeta BD | Disodium EDTA | 0.10 |
| Sodium hydroxide | Sodium hydroxide | 0.18 |
| D-panthenol | Panthenol | 0.50 |
| Dekaben LMB | Iodopropynyl Butylcarbamate | 0.50 |
| Water, demineralized | Aqua | To 100 |

Example 15: O/W Skin Cream

| | INCI/EU | Wt. % |
|---|---|---|
| Oil Phase: | | |
| Emulgin B 3 | Ceteareth-30 | 1.00 |
| Tegin M | Glyceryl stearate | 4.00 |
| Lanette 16 | Cetyl alcohol | 3.00 |
| Beeswax | Cera Alba | 1.00 |
| Softisan 154 | Hydrogenated Palm Oil | 2.00 |
| Cetiol 868 | Octyl stearate | 10.00 |
| Abil 100 | Dimethicone | 1.00 |
| Methyl parabene | Methyl parabene | 0.20 |
| Propyl parabene | Propyl parqabene | 0.05 |
| Perfume | Perfume | 0.50 |
| Aqueous phase: | | |
| Glycerol 86% | Glycerol | 5.00 |
| Phenoxetol | Phenoxyethanol | 0.60 |
| Water, demineralized | Aqua | To 100 |

Example 16: O/W-Body Lotion

| | INCI/EU | Wt. % |
|---|---|---|
| Oil Phase: | | |
| Paraffin oil subliquidum | Paraffinum liquidum | 7.50 |
| Cetagol V | Cetearyl isononanoate | 2.50 |
| Avocado oil | Persea Gratissima | 2.00 |
| Hostaphat 340 D | Trilaureth-4 Phosphate | 3.50 |
| Methyl parabene | Methyl parabene | 0.20 |
| Propyl parabene | Propyl parabene | 0.05 |
| Perfume | Perfume | 0.50 |
| Water phase: | | |
| Carbopol 2984 | Carbomer | 0.30 |
| Sodium hydroxide | Sodium hydroxide | 0.06 |
| Phenoxetol | Phenoxyethanol | 0.60 |
| Glycerol 86% | Glycerol | 5.00 |
| Water, demineralized | Aqua | To 100 |

Example 17: W/O-Body Lotion

| | INCI/EU | Wt. % |
|---|---|---|
| Oil Phase: | | |
| Paraffin oil subliquidum | Paraffinum liquidum | 15.00 |
| Cetiol 868 | Ethylhexyl stearate | 12.00 |
| Dehymuls PGPH | Polyglyceryl-2 dipoly- | 2.00 |
| | hydroxystearate | |
| Zincum N29 | Zinc stearate | 0.50 |
| Vitamin E acetate | Tocopheryl acetate | 0.50 |
| Methyl parabene | Methyl parabene | 0.20 |
| Propyl parabene | Propyl parabene | 0.05 |
| Perfume | Perfume | 0.50 |
| Water phase: | | |
| Zinc sulfate 7-hydrate | Zinc sulfate | |
| Phenoxetol | Phenoxyethanol | 0.60 |
| Glycerol 86% | Glycerol | 8.00 |
| Water, demineralized | Aqua | To 100 |

The disclosure in German Patent Application 102 19 523.4 of May 2, 2002 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a process for production or manufacture of hair or skin cosmetic products using apparatuses with microstructured units, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A process for manufacturing hair cosmetic or skin cosmetic products, wherein said products include a preparation containing at least one hair cosmetic or skin cosmetic ingredient, and said process comprises at least one process step, said at least one process step comprising conducting said preparation, or a part of said preparation, through an apparatus with microstructured units.

2. The process as defined in claim 1, wherein the microstructured units have channels with diameters, each of which is less than or equal to 1000 μm.

3. The process as defined in claim 2, wherein said microstructured units have a plurality of said channels with said diameters of said channels from 10 to 1000 μm and said channels are arranged above each other and/or next to each other, different phases to be mixed flow through said channels and wherein mixing of said different phases occurs during outflow of said phases from said channels.

4. The process as defined in claim 1, wherein said apparatus with said microstructured units is a static micromixer or a microheat exchanger.

5. The process as defined in claim 4, wherein at least one process step comprises thorough mixing of said preparation, or said part of said preparation, with said static micromixer, and further comprising at least one other process step, said at least one other process step including conducting said preparation, or said part of said preparation, through said microheat exchanger.

6. The process as defined in claim 1, wherein said preparation is a microemulsion or a nanoemulsion.

7. The process as defined in claim 1, wherein said hair cosmetic or skin cosmetic products include hair cosmetic or skin cosmetic dispersions, and said process comprises the steps of:
a) feeding a first partial preparation and a second partial preparation not miscible with said first partial preparation into a micromixer;
b) mixing the first partial preparation and the second partial preparation with each other during the feeding of the first partial preparation and the second partial preparation through the micromixer; and
c) supplying said at least one hair cosmetic or skin cosmetic ingredient in one of said first partial preparation and said second partial preparation or adding said at least one hair cosmetic or skin cosmetic ingredient to a mixed preparation resulting from the mixing of step b).

8. The process as defined in claim 7, wherein an aqueous liquid phase is mixed with a hydrophobic liquid phase in the micromixer, said microstructured units have channels each with diameters from 10 to 1000 μm and said mixed preparation or at least one of the aqueous liquid phase and hydrophobic liquid phase is fed to a microheat exchanger, wherein the microheat exchanger has a plurality of said channels with said diameters of from 10 to 1000 μm, and further comprising passing a heat-transfer medium and at least one of the hydrophobic liquid phase, the aqueous liquid phase and the mixed preparation through respective alternate spatially adjacent channels of the microheat exchanger.

9. The process as defined in claim 7, wherein an aqueous liquid phase is mixed with a hydrophobic liquid phase in the micromixer, and one and only one of said aqueous liquid phase and said hydrophobic liquid phase is heated to a temperature above room temperature.

10. The process as defined in claim 9, wherein the hydrophobic liquid phase is a solid at room temperature, is fed to the micromixer in a melted state and the aqueous phase is fed to the micromixer at a temperature less than or equal to room temperature.

11. The process as defined in claim 1, comprising a continuous process, in which a heated mixed preparation prepared in a micromixer is fed to a microheat exchanger and cooled in said microheat exchanger and subsequently a resulting cooled mixed preparation is fed directly into proportioned containers.

12. The process as defined in claim 8, wherein said aqueous liquid phase is mixed with said hydrophobic liquid phase without an emulsifier.

13. The process as defined in claim 9, wherein said hydrophobic liquid phase is liquid at room temperature and is mixed with the aqueous liquid phase at room temperature or below without heating in a micromixer.

14. The process as defined in claim 1, wherein said preparation contains at least one temperature sensitive substance and wherein said preparation or at least one phase or partial preparation containing said at least one temperature sensitive substance is conducted through a microheat exchanger for heating or cooling.

15. The process as defined in claim 14, wherein said at least one temperature sensitive substance is a volatile substance, a substance decomposing at temperatures above room temperature or substances that chemically react with at least one additional ingredient above said room temperature.

16. The process as defined in claim 1, wherein said products include a hair wax product having a waxy consistency at room temperature and containing at least two different waxy effective ingredients or at least one waxy effective ingredient and at least one other non-waxy effective ingredient, and wherein said ingredients of said preparation are conducted through the apparatus with the microstructured units in a heated liquid state and said preparation, after mixing and heating, is conducted through a microheat exchanger for heat transfer.

17. The process as defined in claim 16, wherein said at least one waxy effective ingredient, or one of said at least two different waxy effective ingredients, is a natural wax, fossil wax, synthetic wax, partially synthetic wax, hydrocarbon wax, acid functionalized hydrocarbon wax, polyalkylene glycol wax, polyolefin wax, silicone wax, petrolatum, ozekerite, montan wax, Fischer-Tropsch wax, hardened fat, fatty acid, fatty alcohol, fatty acid ester, fatty alcohol glyceride, or a mono or diester of formula R¹—(C═O)OR², R¹—(C═O)O—R³—O(C═O)R³ and R²O(C═O)—R³—COOR², wherein R¹ represents a C₈- to C₂₂-alkyl group, R² represents a C₃- to C₂₂-alkyl group and R³ represents a C₂- to C₁₆-alkylene group.

18. The process as defined in claim 1, wherein said products include is a hair cleansing or skin cleansing product and said preparation contains at least one wash-active surfactant.

19. The process as defined in claim 18, comprising the steps of:
a) supplying at least one highly concentrated alkyl ether sulfate and an aqueous phase separately to a micromixer;
b) dissolving the alkyl ether sulfate in the aqueous phase during passage through the micromixer to form a resulting solution; and
c) subsequently adding other effective and additive ingredients to the resulting solution.

20. The process as defined in claim 1, wherein said products include a cosmetic sunscreen and said preparation contains at least one light protective effective ingredient and wherein said preparation, or at least a part of said preparation, is conducted through said apparatus with said microstructured units.

21. The process as defined in claim 20, wherein said cosmetic sunscreen comprises an oil or lipid phase and an aqueous phase and said light protective effective ingredient is a UV-light absorbing inorganic pigment, inorganic nano-pigment or an oil-soluble or water-soluble organic UVA-, UVB- or UVA/UVB filtering substance, or a mixture thereof.

22. The process as defined in claim 21, wherein the organic UVA-, UVB- or UVA/UVB filtering substance is at least one member of the group consisting of 2-phenylbenzimidazol-5-sulphonic acid, salts of said 2-phenylbenzimidazol-5-sulphonic acid, cinnamic acid derivatives, silicylic acid derivatives, camphor derivatives, triazine derivatives, benzophenone derivatives, dibenzoylmethane derivatives, β,β-diphenylacrylate derivatives, p-aminobenzoic acid derivatives, menthylanthranilates, polymers with light protecting action and silicones with light protecting action.

23. The process as defined in claim 1, wherein said products include a hair care composition and said hair care composition contains a hydrophilic phase, a hydrophobic phase and at least one effective care ingredient selected from the group consisting of cationic surfactants and C₁₀- to C₃₀-fatty alcohols.

24. The process as defined in claim 23, wherein the hair care composition is creamy and highly viscous.

25. The process as defined in claim 23, wherein said hair care composition is a low viscosity liquid and is sprayable.

26. The process as defined in claim 1, wherein said products include a hair dye composition, and said hair dye composition contains at least one hair coloring substance or at least one oxidation dye precursor product.

27. The process as defined in claim 26, wherein said hair coloring substance is a hair coloring inorganic pigment or a soluble organic direct dye compound for dyeing hair directly.

28. The process as defined in claim 1, wherein said products include a perfumed hair or skin treatment composition, which contains at least one perfume or fragrance.

29. The process as defined in claim 1, wherein said products include a cosmetic skin cream, and said skin cream is an emulsion formed from an aqueous phase and a hydrophobic phase and contains at least one skin care active ingredient.

30. The process as defined in claim 29, wherein said at least one skin care active ingredient is selected from the group consisting of cosmetic oils, vitamins, vitamin derivatives, provitamins, essential fatty acids, sphingolipids, phospholipids, ceramide, betaine and panthenol.

31. The process as defined in claim 1, wherein said preparation contains at least one dispersed powdery solid.

32. The process as defined in claim 31, wherein said at least one dispersed powdery solid is selected from the group consisting of pigments, pearlescence-imparting agents, talcum, mica, kaolin, zinc oxide, titanium oxide, precipitated calcium carbonate, magnesium carbonate, magnesium hydrogen carbonate, silicic acid, glass balls, ceramic balls and powdery polymers.

33. The process as defined in claim 1, wherein the preparation includes at least one ingredient selected from the group consisting of hair care substances, hair cleansing substances, hair-fixing substances, hair coloring substances, light protecting substances, perfumes and preservatives.
